(19) United States
(12) Patent Application Publication
(10) Pub. No.: US 2025/0308689 A1
(43) Pub. Date: Oct. 2, 2025

(54) APPARATUS AND METHOD FOR SYNCHRONIZING VITAL SIGN SIGNALS

(71) Applicant: SAMSUNG ELECTRONICS CO., LTD., Suwon-si (KR)

(72) Inventors: Andriy OLIYNYK, Suwon-si (KR); Illia KRASNOSHCHOK, Suwon-si (KR); Illia FEDORIN, Suwon-si (KR)

(73) Assignee: SAMSUNG ELECTRONICS CO., LTD., Suwon-si (KR)

(21) Appl. No.: 18/745,551

(22) Filed: Jun. 17, 2024

(30) Foreign Application Priority Data
Mar. 26, 2024 (KR) 10-2024-0040897

(51) Int. Cl. G16H 40/67; A61B 5/00; A61B 5/117

(52) U.S. Cl. CPC G16H 40/67; A61B 5/117; A61B 5/4806; A61B 5/7225

(57) ABSTRACT

An apparatus for synchronizing vital sign signals includes a receiver configured to receive a plurality of vital sign signals from a plurality of devices, the plurality of vital sign signals corresponding to a plurality of users, and a processor configured to identify a first user among the plurality of users to whom the plurality of vital sign signals belong based on predetermined circadian rhythm signals of each user of the plurality of users, synchronize vital sign signals corresponding to the first user by matching the vital sign signals corresponding the first user with a first predetermined circadian rhythm signal of the first user, and generate a continuous signal by combining the synchronized vital sign signals.

### CROSS-REFERENCE TO RELATED APPLICATION(S)

This application is based on and claims priority to Korean Patent Application No. 10-2024-0040897, filed on Mar. 26, 2024, in the Korean Intellectual Property Office, the disclosure of which is incorporated by reference herein in its entirety.

### BACKGROUND

### 1. Field

The disclosure relates to an apparatus and method for synchronizing vital sign signals of an individual.

### 2. Description of Related Art

Generally, vital sign signals have very important clinical significance.

Smart home, Internet of Things (IoT) devices and other wearable devices may obtain vital sign signal data about users of the devices and individuals located near the devices, and may transmit the data to servers or terminals to provide information for the users.

Vital sign signals of individuals are personal information that may require protection. If devices are used by a plurality of users, it may be required to accurately identify a user to whom vital sign signals belong, and to synchronize the vital sign signals of the identified user such that the data may be shared among multiple devices.

### SUMMARY

Provided is an apparatus, method, and electronic device capable of synchronizing vital sign signals of a user among a plurality of users.

Additional aspects will be set forth in part in the description which follows and, in part, will be apparent from the description, or may be learned by practice of the presented embodiments of the disclosure.

According to an aspect of the disclosure, an apparatus for synchronizing vital sign signals may include a receiver configured to receive a plurality of vital sign signals from a plurality of devices, the plurality of vital sign signals corresponding to a plurality of users, and a processor configured to identify a first user among the plurality of users to whom the plurality of vital sign signals belong based on predetermined circadian rhythm signals of each user of the plurality of users, synchronize vital sign signals corresponding to the first user by matching the vital sign signals corresponding the first user with a first predetermined circadian rhythm signal of the first user, and generate a continuous signal by combining the synchronized vital sign signals.

The plurality of vital sign signals may include at least one of heart rate, blood pressure, respiratory rate, oxygen saturation, and body temperature.

The predetermined circadian rhythm signals may include a rhythm signal obtained during a period of one day by receiving vital sign signals of each user of the plurality of users.

The processor may be further configured to determine the predetermined circadian rhythm signals based on at least one of standard mathematical analysis, machine learning model, and neural network.

The processor may be configured to identify the first user among the plurality of users to whom the plurality of vital sign signals belongs by comparing the predetermined circadian rhythm signals with the plurality of vital sign signals, and in response to an amplitude difference between the first predetermined circadian rhythm signal and the plurality of vital sign signals being within a threshold range, determining that the plurality of vital sign signals belong to the first user.

The processor may be configured to synchronize the vital sign signals corresponding to the first user by matching at least one of amplitude, phase, and period between the vital sign signals corresponding to the first user with the first predetermined circadian rhythm signal of the first user.

The processor may be further configured to interpolate a missing value in the synchronized vital sign signals of the first user based on the first predetermined circadian rhythm signal of the first user.

The processor may be further configured to transmit the generated continuous signal to the plurality of devices through a communication device.

According to an aspect of the disclosure, a method of synchronizing vital sign signals may include receiving a plurality of vital sign signals from a plurality of devices, the plurality of vital sign signals corresponding to a plurality of users, identifying a first user among the plurality of users to whom the plurality of vital sign signals belong based on predetermined circadian rhythm signals of each user of the plurality of users, synchronizing vital sign signals corresponding to the first user by matching the vital sign signals corresponding to the first user with a first predetermined circadian rhythm signal of the first user, and generating a continuous signal by combining the synchronized vital sign signals.

The plurality of vital sign signals may include at least one of heart rate, blood pressure, respiratory rate, oxygen saturation, and body temperature.

The predetermined circadian rhythm signals may include a rhythm signal obtained during a period of one day by receiving vital sign signals of each user of the plurality of users.

The predetermined circadian rhythm signals may be determined based on at least one of standard mathematical analysis, machine learning model, and neural network.

The identifying of the first user among the plurality of users to whom the plurality of vital sign signals belong may include comparing the predetermined circadian rhythm signals with the plurality of vital sign signals, and in response to an amplitude difference between the first predetermined circadian rhythm signal and the plurality of vital sign signals being within a threshold range, determining that the plurality of vital sign signals belong the first user.

The synchronizing the vital sign signals corresponding to the first user further may include matching at least one of amplitude, phase, and period between the vital sign signals corresponding to the first user with the first predetermined circadian rhythm signal of the first user.

The generating the continuous signal may include interpolating a missing value in the synchronized vital sign signals of the first user based on the first predetermined circadian rhythm signal of the first user.

The method may include transmitting the generated continuous signal to the plurality of devices through a communication device.

According to an aspect of the disclosure, an electronic device may include a main body, and an apparatus provided in the main body and configured to synchronize vital sign signals, where the apparatus includes a receiver configured to receive a plurality of vital sign signals from a plurality of devices, the plurality of vital sign signals corresponding to a plurality of users, and a processor configured to identify a first user among the plurality of users to whom the plurality of vital sign signals belong based on predetermined circadian rhythm signals of each user of the plurality of users, synchronize vital sign signals corresponding to the first user by matching the vital sign signals corresponding the first user with a first predetermined circadian rhythm signal of the first user, and generate a continuous signal by combining the synchronized vital sign signals.

The processor may be further configured to determine the predetermined circadian rhythm signals based on at least one of standard mathematical analysis, machine learning model, and neural network.

The processor may be configured to identify the first user among the plurality of users to whom the plurality of vital sign signals belongs by comparing the predetermined circadian rhythm signals with the plurality of vital sign signals, and in response to an amplitude difference between the first predetermined circadian rhythm signal and the plurality of vital sign signals being within a threshold range, determining that the plurality of vital sign signals belong to the first user.

The electronic device may include a communication device, and the processor may be further configured to transmit the generated continuous signal to the plurality of devices through the communication device.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 is a block diagram illustrating an apparatus for synchronizing vital sign signals according to one or more embodiments;

FIG. 2 is a diagram illustrating an example in which an apparatus for synchronizing vital sign signals receives vital sign signals from a plurality of devices according to one or more embodiments;

FIG. 3A is a graph illustrating a circadian rhythm signal related to a heart rate of a user and a plurality of heart rate signals received by a receiver, according to one or more embodiments;

FIG. 3B is a graph illustrating a circadian rhythm signal related to a blood pressure of a user and a plurality of blood pressure signals received by a receiver, according to one or more embodiments;

FIG. 3C is a graph illustrating a plurality of received blood pressure signals and heart rate signals according to one or more embodiments;

FIG. 4 is a graph illustrating circadian rhythm signal related to a heart rate of a user a and a synchronized heart rate signal of a user, according to one or more embodiments;

FIG. 5 is a flowchart illustrating a method of synchronizing vital sign signals according to one or more embodiments;

FIG. 6 is a diagram illustrating an electronic device including an apparatus for synchronizing vital sign signals, according to one or more embodiments; and

FIGS. 7 to 10 are diagrams illustrating examples of structures of an electronic device including an apparatus for synchronizing vital sign signals, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Hereinafter, example embodiments will be described in detail with reference to the accompanying drawings. The embodiments described below are merely exemplary, and various modifications are possible from these embodiments. In the following drawings, the same reference numerals refer to the same components, and the size of each component in the drawings may be exaggerated for clarity and convenience of description.

In the following description, when a component is referred to as being “above” or “on” another component, it may be directly on an upper, lower, left, or right side of the other component while making contact with the other component or may be above an upper, lower, left, or right side of the other component without making contact with the other component.

Terms such as first, second, etc. may be used to describe various components, but are used only for the purpose of distinguishing one component from another component. These terms do not limit the difference in the material or structure of the components.

The terms of a singular form may include plural forms unless otherwise specified. In addition, when a certain part “includes” a certain component, it means that other components may be further included rather than excluding other components unless otherwise stated.

In addition, terms such as “unit” and “module” described in the specification may indicate a unit that processes at least one function or operation, and this may be implemented as hardware or software, or may be implemented as a combination of hardware and software.

The use of the term “the” and similar designating terms may correspond to both the singular and the plural.

Operations of a method may be performed in an appropriate order unless explicitly described in terms of order. In addition, the use of all illustrative terms (e.g., etc.) is merely for describing technical ideas in detail, and the scope is not limited by these examples or illustrative terms unless limited by the claims.

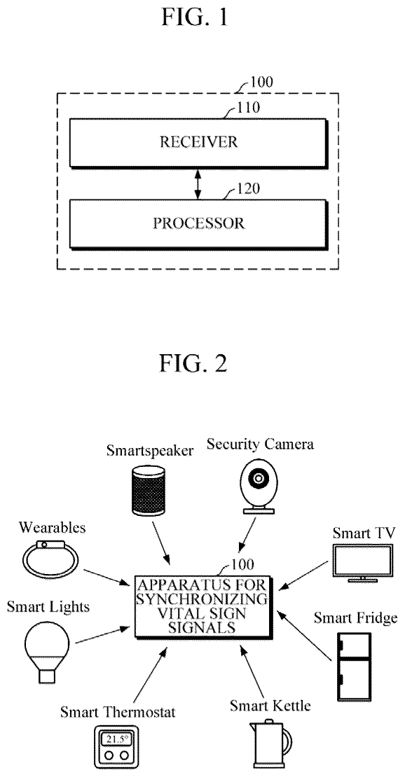

FIG. 1 is a block diagram illustrating an apparatus for synchronizing vital sign signals according to one or more embodiments.

Referring to FIG. 1, an apparatus 100 for synchronizing vital sign signals may include a receiver 110 and a processor 120.

The vital sign signals may include signals indicating whether the physical condition of a human body is controlled within a normal range to maintain homeostasis, and may be personalized vital signs used to assess the physical health of a person. The vital sign signals may include, for example, heart rate, blood pressure, respiratory rate, oxygen saturation, or body temperature, but embodiments are not limited thereto.

The receiver 110 may receive a plurality of vital sign signals from a plurality of devices. For example, the receiver 110 may receive not only vital sign signals obtained by typical sensors mounted in the plurality of devices, but also various vital sign signals obtained through a multi-modal interface.

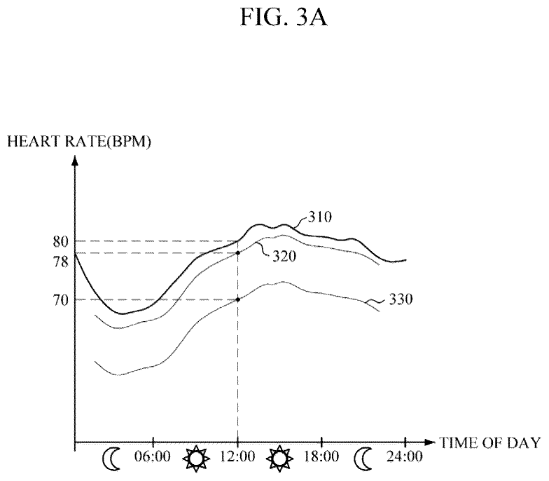

FIG. 2 is a diagram illustrating an example in which an apparatus for synchronizing vital sign signals receives vital sign signals from a plurality of devices according to one or more embodiments.

Referring to FIG. 2, a device for transmitting vital sign signals to the apparatus for synchronizing vital sign signals may include wearable devices (e.g., smart watches, smart rings, smart glasses, etc.), smartphones, cameras, Ultra-Wideband (UWB) Radars, smart televisions (TV), smart fridges, smart lights, smart speakers, smart kettles, smart thermostats, and the like, which may be included in smart home or internet of things (IoT), but embodiments are not limited thereto.

The processor 120 may identify a user to whom the received vital sign signals belong. In this case, the processor 120 may identify a user to whom the received vital sign signals belong, based on a predetermined circadian rhythm signal of each user of a plurality of users.

The circadian rhythm may be a biological rhythm that displays an endogenous oscillation of about 24 hours in plants and animals, and the predetermined circadian rhythm signal of each user may be a circadian rhythm signal obtained during a period of one day by receiving vital sign signals of each user.

The processor 120 may determine the circadian rhythm signal of each user by using at least one of standard mathematical analysis, machine learning model, and neural network, and may store the determined circadian rhythm signal of each user in a storage and the like.

For example, the processor 120 may receive heart rate signals at various times from a plurality of devices used by users, and may obtain circadian rhythm signals related to heart rate based on a neural network by using the received heart rate signals as an input. The processor 120 may directly obtain circadian rhythm signals from a plurality of devices used by users. However, the method of obtaining circadian rhythm signals is not limited thereto.

The processor 120 may compare the predetermined circadian rhythm signal of each user with the received vital sign signals to identify a user to whom the received vital sign signals belong. For example, the processor 120 may compare the circadian rhythm signal of each user with received vital sign signals, and if an amplitude difference between the signals (i.e., between the received vital sign signals and the predetermined circadian rhythm signals) is within a threshold range, the processor 120 may determine that the signals belong to the same user. That is, if an amplitude difference between the circadian rhythm signal and the vital sign signals is within a threshold range, the processor 120 may determine that the circadian rhythm signal and the vital sign signals are signals of the same user.

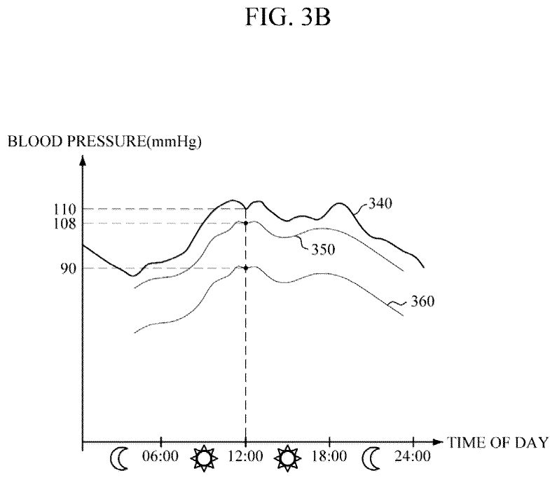

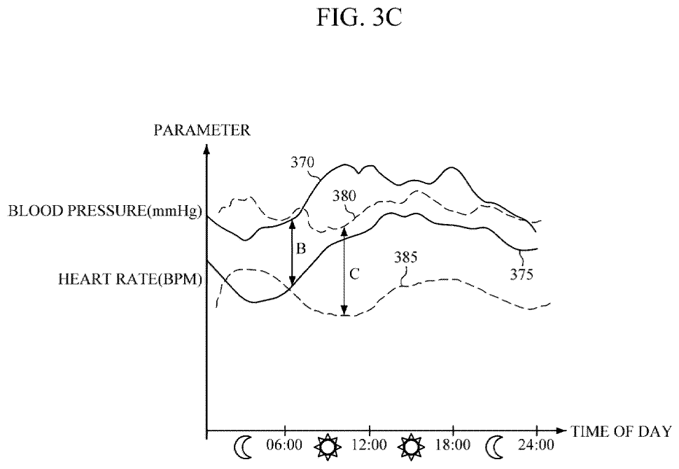

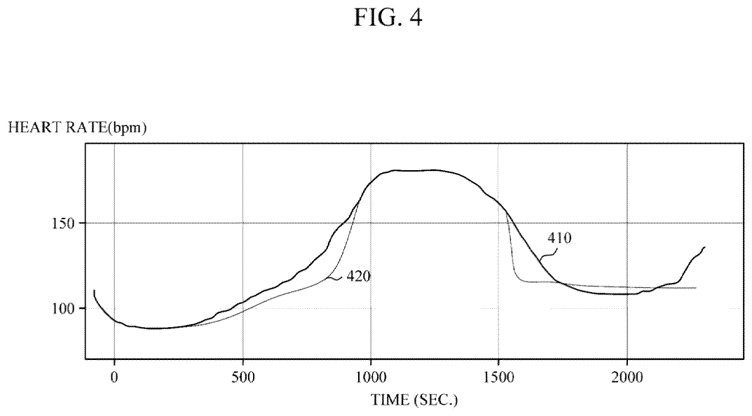

FIG. 3A is a graph illustrating a circadian rhythm signal related to a heart rate of a user and a plurality of heart rate signals received by a receiver, according to one or more embodiments. That is, FIG. 3A is a diagram illustrating a circadian rhythm signal related to the heart rate of user A and a plurality of heart rate signals received by the receiver 110. FIG. 3B is a graph illustrating a circadian rhythm signal related to a blood pressure of a user and a plurality of blood pressure signals received by a receiver, according to one or more embodiments. That is, FIG. 3B is a diagram illustrating a circadian rhythm signal related to the blood pressure of user A and a plurality of blood pressure signals received by the receiver 110.

Referring to FIG. 3A, the processor 120 may determine, for example, 4 beats per minute (BPM) as a threshold range of heart rate for determining whether signals belong to user A.

In this case, upon comparing a circadian rhythm signal 310 (e.g., a predetermined circadian rhythm signal) related to the heart rate of user A with a received heart rate signal 320, an amplitude difference between the signals 310 and 320 is within the threshold range. Specifically, the heart rate in the circadian rhythm signal 310 at noon is 80 BPM, and the heart rate in the heart rate signal 320 at noon is 78 BPM, such that the difference therebetween is within the threshold range of 4 BPM. An overall difference between the heart rate signal 320 and the circadian rhythm signal 310 falls within the range of 4 BPM. Accordingly, the processor 120 may determine that the heart rate signal 320 is the heart rate signal of user A, even though there is a difference between the predetermined circadian rhythm signal 310 and the received heart rate signal 320.

In addition, upon comparing the circadian rhythm signal 310 related to the heart rate of user A with a received heart rate signal 330, an amplitude difference between the signals 310 and 330 falls outside a threshold range. That is, the heart rate in the circadian rhythm signal 310 at noon is 80 BPM, and the heart rate in the heart rate signal 330 at noon is 70 BPM, such that the difference therebetween falls outside the threshold range of 4 BPM. An overall difference between the heart rate signal 330 and the circadian rhythm signal 310 falls outside the range of 4 BPM. Accordingly, the processor 120 may determine that the heart rate signal 330 is the heart rate signal of another user that is not user A.

Referring to FIG. 3B, the processor 120 may determine, for example, 5 mmHg (i.e., blood pressure) as a threshold range of blood pressure for determining whether signals belong to the same user.

In this case, upon comparing a circadian rhythm signal 340 (e.g., a predetermined circadian rhythm signal) related to the blood pressure of user A with a received blood pressure signal 350, an amplitude difference between the signals 340 and 350 is within the threshold range. Specifically, the blood pressure in the circadian rhythm signal 340 at noon is 110 mmHg, and the blood pressure in the blood pressure signal 350 at noon is 108 mmHg, such that the difference therebetween is within the threshold range of 5 mmHg. An overall difference between the blood pressure signal 350 and the circadian rhythm signal 340 falls within the range of 5 mmHg. Accordingly, the processor 120 may determine that the blood pressure signal 350 is the blood pressure signal of user A, even though there is a difference between the predetermined circadian rhythm signal 340 and the received blood pressure signal 350.

In addition, upon comparing the circadian rhythm signal 340 related to the blood pressure of user A with a received blood pressure signal 360, an amplitude difference between the signals 340 and 360 falls outside a threshold range. That is, the blood pressure in the circadian rhythm signal 340 at noon is 110 mmHg, and the blood pressure in the blood pressure signal 360 at noon is 90 mmHg, such that the difference therebetween falls outside the threshold range of 5 mmHg. The overall difference between the blood pressure signal 360 and the circadian rhythm signal 340 falls outside the range of 5 mmHg. Accordingly, the processor 120 may determine that the blood pressure signal 360 is the blood pressure signal of another user that is not user A.

In another example, the processor 120 may also determine whether signals belong to the same user by using different types of vital sign signals. For example, if an amplitude difference between a blood pressure signal and a heart rate signal is within a threshold range, the processor 120 may determine that the blood pressure signal and the heart rate signal are signals of the same user.

FIG. 3C is a graph illustrating a plurality of received blood pressure signals and heart rate signals according to one or more embodiments. That is, FIG. 3C is a diagram illustrating a plurality of received blood pressure signals and heart rate signals, in which the X-axis represents time of day, and the Y-axis represents blood pressure and heart rate.

Referring to FIG. 3C, the processor 120 may determine, for example, a predetermined amplitude difference X for determining that a blood pressure signal and a heart rate signal are signals of the same user.

In this case, if an amplitude difference B between a blood pressure signal 370 and a heart rate signal 375 is equal to or smaller than the predetermined amplitude difference X (X≥B), the processor 120 may determine that the blood pressure signal 370 and the heart rate signal 375 are signals of the same user, and if an amplitude difference C between a blood pressure signal 380 and a heart rate signal 385 is equal to or greater than the predetermined amplitude difference X (X<C), the processor 120 may determine that the blood pressure signal 380 and the heart rate signal 385 are signals of different users.

The processor 120 may synchronize the vital sign signals of the identified user by matching the vital sign signals of the identified user with the circadian rhythm signal of the same user. In this case, the processor 120 may synchronize the vital sign signals by matching at least one of amplitude, phase, and period between the vital sign signals of the same user and the circadian rhythm signal of the same user.

For example, upon determining that received blood pressure signals, which are measured between 2:30 PM and 2:35 PM by a smartwatch, are blood pressure signals of user A, the processor 120 may match a circadian rhythm signal related to blood pressure of user A with the received blood pressure signals to synchronize the received blood pressure signals by, for example, adjusting an amplitude of the received blood pressure signals according to predetermined conditions, matching a phase angle or a period thereof, etc., in comparison with the circadian rhythm signal related to blood pressure.

The processor 120 may generate a continuous signal by combining the synchronized vital sign signals. For example, the processor 120 may generate one continuous signal by combining synchronized blood pressure signals of user A, which are received from various devices, including, for example, synchronized blood pressure signals of user A, which are received from a smartwatch, synchronized blood pressure signals of user A which are received from a smartphone, and the like.

The processor 120 may store the generated signal in a storage, may provide a user with bio-information in response to a user's request, and may transmit the generated signal to each of the devices that transmitted vital sign signals, through a communication device.

If there is a missing value when combining the synchronized vital sign signals, the processor 120 may interpolate the missing value by using the circadian rhythm signal of the same user.

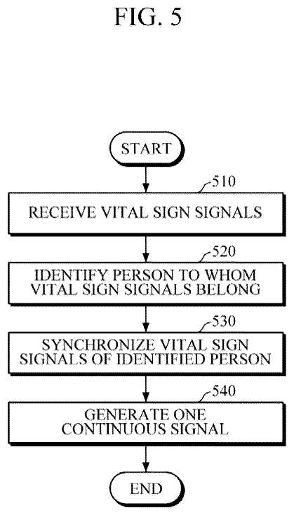

FIG. 4 is a graph illustrating circadian rhythm signal related to a heart rate of a user a and a synchronized heart rate signal of a user, according to one or more embodiments. That is, FIG. 4 is a circadian rhythm signal related to the heart rate of user A and a synchronized heart rate signal of user A.

Referring to FIG. 4, there is a missing value in a synchronized heart rate signal 420 of user A during a period between about 1000 and 1500 seconds. In this case, the processor 120 may interpolate the missing value by using a value of a circadian rhythm signal 410 (e.g., a predetermined circadian rhythm signal of user A) related to heart rate during the period between about 1000 and 1500 seconds. The missing value may be interpolated by using not only the circadian rhythm signal but also a statistical value (e.g., mean) of vital sign signals obtained from other devices, and the like.

In an environment using multiple wired and wireless devices, accurately identifying and synchronizing vital sign signals of users in terms of user information protection may be important. In one or more embodiments, by identifying a user to whom vital sign signals belong based on a predetermined or otherwise generated circadian rhythm signal of each user, and by synchronizing the vital sign signals of the same user to generate a continuous signal, unnecessary memory consumption for identifying and synchronizing signals may be reduced, personal information may be protected, and accurate information about vital sign signals may be provided to users.

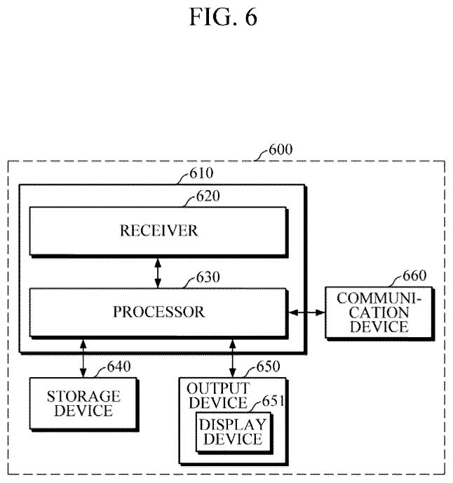

FIG. 5 is a flowchart illustrating a method of synchronizing vital sign signals according to one or more embodiments.

The method of FIG. 5 is an example of a method of synchronizing vital sign signals performed by the apparatus 100 for synchronizing vital sign signals according to one or more embodiments. Descriptions redundant with those above may be omitted.

Referring to FIG. 5, the apparatus for synchronizing vital sign signals may receive a plurality of vital sign signals from a plurality of devices in operation 510. The vital sign signals may include at least one of heart rate, blood pressure, respiratory rate, oxygen saturation, and body temperature, but embodiments are not limited thereto.

The apparatus for synchronizing vital sign signals may identify a user to whom the received vital sign signals belong, based on predetermined circadian rhythm signals of each user in operation 520. The predetermined circadian rhythm signals of each user may be circadian rhythm signals obtained during one day by receiving vital sign signals of each user. Each user may refer to a plurality of users that may interact with the plurality of devices within the environment. The predetermined circadian rhythm signals of each user may be obtained by using at least one of standard mathematical analysis, machine learning model, and neural network.

The apparatus for synchronizing vital sign signals may compare the predetermined circadian rhythm signals of each user with the received vital sign signals, and if an amplitude difference between the predetermined circadian rhythm signals of each user and the received vital sign signals is within a threshold range, the apparatus for synchronizing vital sign signals may determine that a first predetermined circadian rhythm signal and the received vital sign signals are signals of the same user. That is, the apparatus may obtain a plurality of predetermined circadian rhythm signals respectively corresponding to a plurality of users, such as user A1, user A2, user A3, etc., and then may compare the received plurality of vital sign signals with the plurality of predetermined circadian rhythm signals. Based on at least one of the plurality of predetermined circadian rhythm signals being within a predetermined threshold of the received vital sign signals, the apparatus may determine the at least one predetermined circadian rhythm signal (i.e., a first predetermined circadian rhythm signal) corresponds to one of the users (i.e., user A1) of the plurality of users.

The apparatus for synchronizing vital sign signals may synchronize the vital sign signals of the user (i.e., user A1) by matching the vital sign signals of the user with the circadian rhythm signal of the same user in operation 530. For example, the apparatus for synchronizing vital sign signals may synchronize the vital sign signals by matching at least one of amplitude, phase, and period between the vital sign signals of the same user and the circadian rhythm signal of the same user. That is, when user A1 is determined to be the matched user, the apparatus may match the vital sign signals with the first predetermined circadian rhythm signal that corresponds to user A1.

The apparatus for synchronizing vital sign signals may combine the synchronized vital sign signals to generate one continuous signal in operation 540. If there is a missing value in the synchronized vital sign signals, the apparatus for synchronizing vital sign signals may interpolate the missing value by using the circadian rhythm signal of the user. The apparatus for synchronizing vital sign signals may further include a communication device, and may transmit the generated one continuous signal to a plurality of devices through the communication device.

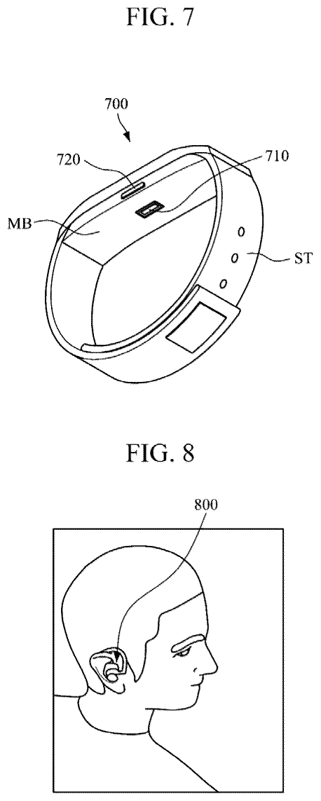

FIG. 6 is a diagram illustrating an electronic device including an apparatus for synchronizing vital sign signals, according to one or more embodiments.

The electronic device may be, for example, various types of wearable devices, such as smart watches, smart bands, smart glasses, smart earphones, smart rings, smart patches, and smart necklaces, and mobile devices such as smartphones, tablet personal computers (PCs), etc., or various IoT devices (e.g., home IoT devices, etc.). In addition, the electronic device may be a local or cloud server, but embodiments are not limited thereto.

Referring to FIG. 6, a main body 600 of the electronic device may include an apparatus 610 for synchronizing vital sign signals, a storage device 640, an output device 650, and a communication device 660. The apparatus 610 for synchronizing vital sign signals may include a receiver 620 and a processor 630.

The receiver 620 may receive a plurality of vital sign signals from a plurality of devices. The vital sign signals may include at least one of heart rate, blood pressure, respiratory rate, oxygen saturation, and body temperature.

The processor 630 may identify a user to whom the received vital sign signals belong based on a predetermined circadian rhythm signal of each user among a plurality of users within the environment, may synchronize the vital sign signals of the identified user by matching the vital sign signals with the circadian rhythm signal of the user, and may combine the synchronized vital sign signals to generate a continuous signal.

In addition, the processor 630 may determine the circadian rhythm signal of each user by using at least one of standard mathematical analysis, machine learning model, and neural network, and may compare the circadian rhythm signal of each user with received vital sign signals. If an amplitude difference between the signals is within a threshold range, the processor 630 may determine that the signals belong to the same user.

The storage device 640 may store information related to synchronizing vital sign signals. For example, the storage device 640 may store circadian rhythm signals of each user, machine learning models and/or neural network models for obtaining the circadian rhythm signals of each user, and the like.

The storage device 640 may include a storage medium having at least one type of a flash memory type, a hard disk type, a multimedia card micro type, a card type (e.g., a secure digital (SD) memory, a XD memory, etc.), a random access memory (RAM), a static RAM (SRAM), a read-only memory (ROM), an electrically erasable programmable ROM (EEPROM), a programmable ROM (PROM), a magnetic memory, a magnetic disk, or an optical disk, etc., but embodiments are not limited thereto.

The output device 650 may provide processing results of the processor 630 to a user. For example, the output device 650 may display circadian rhythm signals or vital sign signals of a user on the display device 651. In this case, if a vital sign signal value falls outside a normal range, the output device 650 may provide the user with warning information by changing color, line thickness, etc., or by displaying the abnormal value along with the normal range, so that the user may easily recognize the signal value.

The display device 651 may include, for example, a display, a hologram device, or a projector and control circuitry to control the devices. The display device 651 may include touch circuitry adapted to detect a touch, and/or sensor circuitry (e.g., pressure sensor, etc.) adapted to measure the intensity of force incurred by the touch.

The communication device 660 may communicate with an external device to transmit and receive various data related to synchronizing vital sign signals. For example, the communication device 660 may transmit one continuous signal, generated by the apparatus for synchronizing vital sign signals, to another electronic device.

The communication device 660 may communicate with the external device by using various wired and wireless communication techniques including Bluetooth communication, Bluetooth low energy (BLE) communication, near field communication (NFC), WLAN communication, Zigbee communication, Infrared Data Association (IrDA) communication, Wi-Fi Direct (WFD) communication, ultra-wideband (UWB) communication, Ant+ communication, WIFI communication, radio frequency identification (RFID) communication, 3^{rd} generation (3G), 4^{th} generation (4G), 5^{th} generation (5G), and 6^{th} generation (6G) communications, and the like. However, the communication techniques are not limited thereto.

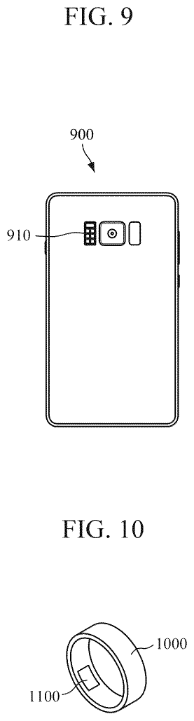

FIGS. 7 to 10 are diagrams illustrating examples of structures of an electronic device including the apparatus described above for synchronizing vital sign signals, according to one or more embodiments.

Referring to FIG. 7, the electronic device may be implemented as a smartwatch-type wearable device 700 which includes a main body MB and a wrist strap ST.

The main body MB may be formed in various shapes. A battery may be embedded in the main body MB and/or the strap ST to supply power to various components of the wearable device.

The strap ST may be connected to both ends of the main body to allow the main body to be worn on a user's wrist, and may be flexible so as to be wrapped around the user's wrist. The strap ST may be composed of a first strap and a second strap which are separated from each other. One ends of the first and second straps are connected to both sides of the main body MB, and the first and second straps may be connected to each other via a fastening mechanism formed at the other ends of the first and second straps. The fastening mechanism may be formed as magnetic fastening, Velcro fastening, pin fastening, etc., but embodiments are not limited thereto. Further, the strap ST is not limited thereto, and may be integrally formed as a non-detachable band.

The main body MB may include the apparatus for synchronizing vital sign signals, a display device, an output device, a storage device, a communication device, and the like. However, depending on the size and shape of a form factor and the like, some of the display device, the output device, the storage device, and the communication chip may be omitted.

A sensor 710 may be mounted on a rear surface of the main body MB, so that when the main body MB is worn on a user's wrist, the sensor 710 may contact the upper part of the user's wrist to obtain vital sign signal data from the wrist. In addition to the rear surface of the main body MB, the sensor 710 may also be mounted on the wrist strap ST to obtain data.

A manipulator 720 may be formed on a side surface of the main body MB, as illustrated herein. The manipulator 720 may receive a user command and transmit the user command to the processor. In addition, the manipulator 720 may have a power button to turn on/off the wearable device 700.

The apparatus for synchronizing vital sign signals, which is mounted in the main body MB, may include a receiver and a processor. The receiver may receive a plurality of vital sign signals from a plurality of devices, and the processor may identify a user to whom the received vital sign signals belong based on a predetermined circadian rhythm signal of each user, may synchronize the vital sign signals of the identified user by matching the vital sign signals of the identified user with the circadian rhythm signal of the same user, and may combine the synchronized vital sign signals to generate a continuous signal.

A display may be provided on a front surface of the main body MB and may display circadian rhythm signals or vital sign signals of a user. In this case, if a vital sign signal value falls outside a normal range, the display may provide the user with warning information by changing color, line thickness, etc., or by displaying the abnormal value along with the normal range, so that the user may easily recognize the signal value.

Referring to FIG. 8, the electronic device may also be implemented as an ear-wearable device 800.

The ear-wearable device 800 may include a main body and an ear strap. A user may wear the ear-wearable device 800 by wearing the ear strap on the auricle. The ear strap may be omitted depending on the shape of the ear-wearable device 800. The main body may be inserted into the external auditory meatus. An apparatus for synchronizing vital sign signals, a sensor, a display device, an output device, a storage device, a communication device, and the like may be provided in the main body.

The ear-wearable device 800 may provide a user with results of synchronizing vital sign signals in the form of sounds, or may transmit the results to an external device, e.g., a user's mobile device, tablet PC, PC, etc., through the communication device provided in the main body.

Referring to FIG. 9, the electronic device may be implemented as a mobile device 900 such as a smartphone.

The mobile device 900 may include a housing and a display panel. The housing may form the exterior of the mobile device 900. The housing has a first surface, on which a display panel and a cover glass may be disposed sequentially, and the display panel may be exposed to the outside through the cover glass. A sensor 910, a camera module and/or an infrared sensor, and the like may be mounted on a second surface of the housing.

For example, a plurality of sensors for obtaining data from a user may be mounted on a rear surface of a main body of the mobile device 900, including a fingerprint sensor mounted on a front surface of the mobile device 900, sensors disposed at a power button or a volume button on a side surface thereof or disposed at other positions of the front and rear surfaces of the mobile device 900, and the like.

In addition, if a user transmits a request for synchronizing vital sign signals by executing an application and the like installed in the mobile device 900, the mobile device 900 may obtain vital sign signal data from another device by using the receiver, and may identify a user and synchronize the vital sign signals by using the processor.

Referring to FIG. 10, the electronic device may be implemented as a ring-type wearable device 1000.

The ring-type wearable device 1000 may include an apparatus for synchronizing vital sign signals, a display device, an output device, a storage device, and a communication device, and the apparatus for synchronizing vital sign signals may include a receiver and a processor.

The receiver may receive a plurality of vital sign signals from a plurality of devices, and the processor may identify a user to whom the received vital sign signals belong based on a predetermined circadian rhythm signal of each user among a plurality of users in the environment, may synchronize the vital sign signals of a first user by matching the vital sign signals of the first user with the circadian rhythm signal of the first user, and may combine the synchronized vital sign signals to generate a continuous signal.

When a main body of the ring is worn on a user's finger, a sensor 1100 may obtain vital sign signal data from the finger.

One or more embodiments may be realized as a computer-readable code written on a computer-readable recording medium. The computer-readable recording medium may be any type of recording device in which data is stored in a computer-readable manner.

As used in connection with various embodiments of the disclosure, the term “module” may include a unit implemented in hardware, software, or firmware, and may interchangeably be used with other terms, for example, logic, logic block, part, or circuitry. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software including one or more instructions that are stored in a storage medium that is readable by a machine. For example, a processor of the machine may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

At least one of the devices, units, components, modules, units, or the like represented by a block or an equivalent indication in the above embodiments including, but not limited to, FIGS. 1, 2 and 6, may be physically implemented by analog and/or digital circuits including one or more of a logic gate, an integrated circuit, a microprocessor, a microcontroller, a memory circuit, a passive electronic component, an active electronic component, an optical component, and the like, and may also be implemented by or driven by software and/or firmware (configured to perform the functions or operations described herein).

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An apparatus for synchronizing vital sign signals, the apparatus comprising:
a receiver configured to receive a plurality of vital sign signals from a plurality of devices, the plurality of vital sign signals corresponding to a plurality of users; and
a processor configured to:
identify a first user among the plurality of users to whom the plurality of vital sign signals belong based on predetermined circadian rhythm signals of each user of the plurality of users;
synchronize vital sign signals corresponding to the first user by matching the vital sign signals corresponding the first user with a first predetermined circadian rhythm signal of the first user; and
generate a continuous signal by combining the synchronized vital sign signals.

2. The apparatus of claim 1, wherein the plurality of vital sign signals comprise at least one of heart rate, blood pressure, respiratory rate, oxygen saturation, and body temperature.

3. The apparatus of claim 1, wherein the predetermined circadian rhythm signals comprise a rhythm signal obtained during a period of one day by receiving vital sign signals of each user of the plurality of users.

4. The apparatus of claim 3, wherein the processor is further configured to determine the predetermined circadian rhythm signals based on at least one of standard mathematical analysis, machine learning model, and neural network.

5. The apparatus of claim 1, wherein the processor is configured to identify the first user among the plurality of users to whom the plurality of vital sign signals belongs by:
comparing the predetermined circadian rhythm signals with the plurality of vital sign signals; and
in response to an amplitude difference between the first predetermined circadian rhythm signal and the plurality of vital sign signals being within a threshold range, determining that the plurality of vital sign signals belong to the first user.

6. The apparatus of claim 1, wherein the processor is configured to synchronize the vital sign signals corresponding to the first user by matching at least one of amplitude, phase, and period between the vital sign signals corresponding to the first user with the first predetermined circadian rhythm signal of the first user.

7. The apparatus of claim 1, wherein the processor is further configured to interpolate a missing value in the synchronized vital sign signals of the first user based on the first predetermined circadian rhythm signal of the first user.

8. The apparatus of claim 1, wherein the processor is further configured to transmit the generated continuous signal to the plurality of devices through a communication device.

9. A method of synchronizing vital sign signals, the method comprising:
receiving a plurality of vital sign signals from a plurality of devices, the plurality of vital sign signals corresponding to a plurality of users;
identifying a first user among the plurality of users to whom the plurality of vital sign signals belong based on predetermined circadian rhythm signals of each user of the plurality of users;
synchronizing vital sign signals corresponding to the first user by matching the vital sign signals corresponding to the first user with a first predetermined circadian rhythm signal of the first user; and
generating a continuous signal by combining the synchronized vital sign signals.

10. The method of claim 9, wherein the plurality of vital sign signals comprise at least one of heart rate, blood pressure, respiratory rate, oxygen saturation, and body temperature.

11. The method of claim 9, wherein the predetermined circadian rhythm signals comprise a rhythm signal obtained during a period of one day by receiving vital sign signals of each user of the plurality of users.

12. The method of claim 11, wherein the predetermined circadian rhythm signals are determined based on at least one of standard mathematical analysis, machine learning model, and neural network.

13. The method of claim 9, wherein the identifying of the first user among the plurality of users to whom the plurality of vital sign signals belong comprises:
comparing the predetermined circadian rhythm signals with the plurality of vital sign signals; and
in response to an amplitude difference between the first predetermined circadian rhythm signal and the plurality of vital sign signals being within a threshold range, determining that the plurality of vital sign signals belong the first user.

14. The method of claim 9, wherein the synchronizing the vital sign signals corresponding to the first user further comprises matching at least one of amplitude, phase, and period between the vital sign signals corresponding to the first user with the first predetermined circadian rhythm signal of the first user.

15. The method of claim 9, wherein the generating the continuous signal comprises interpolating a missing value in the synchronized vital sign signals of the first user based on the first predetermined circadian rhythm signal of the first user.

16. The method of claim 9, further comprising transmitting the generated continuous signal to the plurality of devices through a communication device.

17. An electronic device comprising:
a main body; and
an apparatus provided in the main body and configured to synchronize vital sign signals,
wherein the apparatus comprises:
a receiver configured to receive a plurality of vital sign signals from a plurality of devices, the plurality of vital sign signals corresponding to a plurality of users; and
a processor configured to:
identify a first user among the plurality of users to whom the plurality of vital sign signals belong based on predetermined circadian rhythm signals of each user of the plurality of users;
synchronize vital sign signals corresponding to the first user by matching the vital sign signals corresponding the first user with a first predetermined circadian rhythm signal of the first user; and
generate a continuous signal by combining the synchronized vital sign signals.

18. The electronic device of claim 17, wherein the processor is further configured to determine the predetermined circadian rhythm signals based on at least one of standard mathematical analysis, machine learning model, and neural network.

19. The electronic device of claim 17, wherein the processor is configured to identify the first user among the plurality of users to whom the plurality of vital sign signals belongs by:
comparing the predetermined circadian rhythm signals with the plurality of vital sign signals; and
in response to an amplitude difference between the first predetermined circadian rhythm signal and the plurality of vital sign signals being within a threshold range, determining that the plurality of vital sign signals belong to the first user.

20. The electronic device of claim 17, further comprising a communication device,
wherein the processor is further configured to transmit the generated continuous signal to the plurality of devices through the communication device.
